(19) United States
(12) Patent Application Publication
(10) Pub. No.: US 2019/0090722 A1
(43) Pub. Date: Mar. 28, 2019

(54) MEDICAL INSTRUMENT

(71) Applicant: CANON KABUSHIKI KAISHA, Tokyo (JP)

(72) Inventor: Takahisa Kato, Brookline, MA (US)

(21) Appl. No.: 16/203,423

(22) Filed: Nov. 28, 2018

Related U.S. Application Data
(63) Continuation of application No. 14/403,776, filed on Nov. 25, 2014.

(30) Foreign Application Priority Data
May 31, 2012 (JP) 2012-124504

(51) Int. Cl. A61B 1/005; A61B 1/05; A61B 5/04; A61B 1/00; A61B 1/06; A61B 5/00; A61B 1/07; A61M 25/01

(52) U.S. Cl. CPC A61B 1/0057; A61B 1/05; A61B 5/04; A61B 1/00006; A61M 25/0147; A61B 1/0016; A61B 5/6852; A61B 1/07; A61B 1/0052; A61B 1/0676

(57) ABSTRACT

When a bendable medical instrument is used, the present invention reduces a risk that a body of the medical instrument is damaged.

The present invention provides a medical instrument which includes: a deformable portion; a wire configured to deform the deformable portion; and a driving unit configured to transmit driving force to the wire, wherein: the medical instrument includes a load detecting unit configured to detect load applied to the deformable portion; and when the load detected by the load detecting unit exceeds a threshold value, the driving unit breaks connection between the wire and the driving force.

### CROSS REFERENCE TO RELATED APPLICATIONS

The present application is a continuation of U.S. patent application Ser. No. 14/403,776, filed on Nov. 25, 2014, which is a National Phase Application of International Application PCT/JP2013/064561 filed May 21, 2013, which claims priority from Japanese Patent Application No. 2012-124504 filed May 31, 2012, which is hereby incorporated by reference herein in its entirety.

### TECHNICAL FIELD

The present invention relates to a medical instrument, such as an endoscope and a catheter, which is capable of being bent and guided.

### BACKGROUND ART

A medical device, such as an endoscope and an electrophysiological catheter, which passes through a structure of a living body, such as a body cavity, and accesses a target location includes an inserting portion which is inserted in a patient's body. Some medical devices include a bendable bending portion in the inserting portion which may follow the structure of the living body. The success rate of inspection and medical care may be increased by guiding the device to various locations of the living body using a bending function.

In such a related art device, an operation wire is attached to a bendable structure and, when the operation wire is drawn by a driving unit, a bending operation is performed.

If the bending operation is performed inside the body cavity, it is necessary to consider contact of the device with the body cavity or peripheral structures thereof and to consider a harmful effect caused by the contact. There has also been a rigid endoscope which may detect contact with a body cavity. PTL 1 describes an invention related to a retreat of a bendable endoscope by bending and a process in a case in which external load is applied to a treatment tool in which a sheath like the endoscope is used.

When a bendable medical device is used, there is a possibility that damage is caused to a medical instrument due to overload if excessively large load is applied to the inserting portion because, for example, a thin material is used in a small-sized endoscope.

### CITATION LIST

### Patent Literature

PTL 1 Japanese Patent Laid-Open No. 2010-175962

### SUMMARY OF INVENTION

The present invention provides a medical instrument which is capable of reducing damage, such as cutting of a wire, to the medical instrument even if excessively large load is applied to an inserting portion.

The present invention provides a medical instrument which includes: a deformable portion; a wire configured to deform the deformable portion; and a driving unit configured to transmit driving force to the wire, wherein: the medical instrument includes a load detecting unit configured to detect load applied to the deformable portion; and when the load detected by the load detecting unit exceeds a threshold value, the driving unit breaks connection between the wire and the driving force.

Further features of the present invention will become apparent from the following description of exemplary embodiments with reference to the attached drawings.

### BRIEF DESCRIPTION OF DRAWINGS

FIG. 1A is a side view of a medical instrument according to one embodiment of the present invention and

FIGS. 1B and 1C are diagrams illustrating a bending operation of a medical base portion according to one embodiment of the present invention.

FIG. 2 is a block diagram illustrating a configuration of a medical instrument according to one embodiment of the present invention.

FIG. 3A is a side view illustrating a state in which a medical instrument according to one embodiment of the present invention is in contact with an environment with an inserting portion thereof being bent, and

FIG. 3B is a side view illustrating a state in which an inserting portion of the medical instrument according to one embodiment of the present invention is in contact with the environment with the environment being moved.

FIG. 4 is a flowchart illustrating an operation of the medical instrument according to one embodiment of the present invention.

FIG. 5 is a conceptual diagram which simulates elasticity of an inserting portion of a medical instrument according to one embodiment of the present invention.

FIG. 6 is a side view illustrating another embodiment of a medical instrument according to the present invention.

FIG. 7 is a block diagram illustrating a third embodiment of a medical instrument of the present invention.

FIG. 8 is a block diagram illustrating a fourth embodiment of a medical instrument of the present invention.

FIG. 9 is a schematic cross-sectional view of a tip portion of a medical instrument according to one embodiment of the present invention.

### DESCRIPTION OF EMBODIMENTS

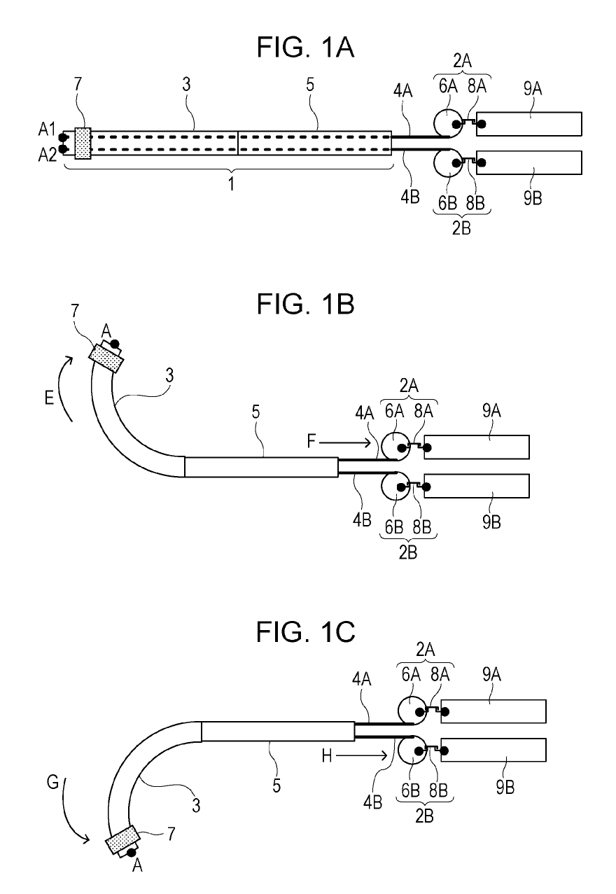

As illustrated in FIGS. 1A to 1C, a medical instrument includes a bending portion 3 which is a deformable portion, wires 4A and 4B which deform the deformable portion (hereafter, referred to as “control wires”), drive transmitting units 2A and 2B which transmit driving force to the wires, and a tactile sensor 7 which detects load applied to, for example, the deformable portion. Each component is controlled by a drive control unit which controls driving.

Using an inserting portion 1 which includes the deformable portion, a medical instrument, such as an endoscope and a catheter for observing inside a human body or inside a structure which cannot be directly observed may be provided.

If load detected by the load detecting unit exceeds a threshold value, the drive transmitting units break connection between the wires and the driving force. Breaking the connection between the wires and the driving force means stopping transmission of the force to the wires. Then, the wires are put into a state in which no driving force is applied thereto.

Breaking the connection between the wires and the driving force may be stopping of the driving force transmitted to the wires or may be physical disconnection of the drive transmitting units which transmit the driving force to the wires.

Since the drive transmitting units may stop the supply of the driving force to the wires, the drive transmitting units may be referred also to as driving force stopping units.

Preferably, a load control unit includes a measuring unit for measuring force from outside. Exemplary measuring units include a measuring unit which measures pressure, a measuring unit which measures a current and a measuring unit which measures tension.

Since excessively large load may be prevented from being applied to the wires by breaking the connection between the wires and the driving force, the risk of cutting of the wires may be reduced.

If excessively large load is applied to the wires, the load is often applied also to the inserting portion. Therefore, controlling application of excessively large load to the wires leads to reduction of damage to the inserting portion.

### First Embodiment

Hereinafter, a preferred embodiment of the medical instrument according to the invention will be described.

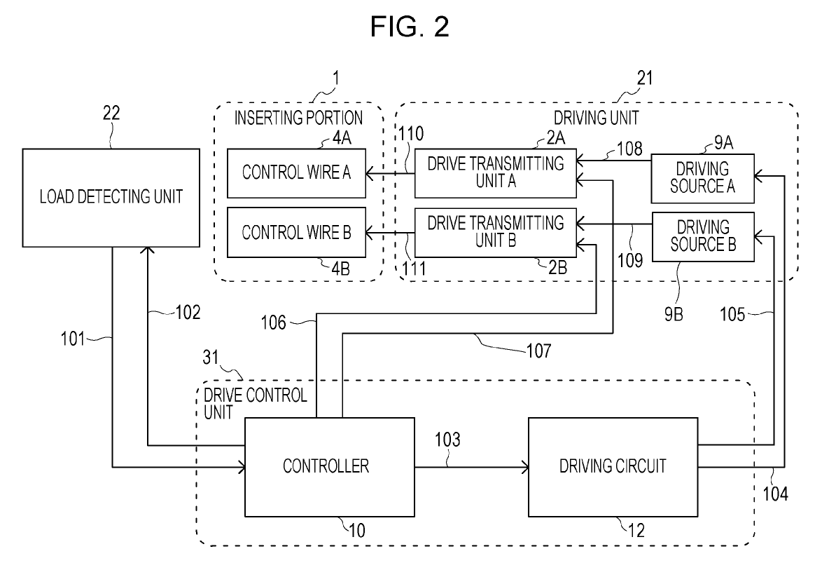

The medical instrument according to the present embodiment includes a configuration illustrated in FIGS. 1A to 1C and 2. FIGS. 1A to 1C are side views illustrating an inserting portion and a driving unit of the medical instrument according to the present embodiment.

FIG. 2 is a block diagram illustrating a configuration of the medical instrument according to the present embodiment. The medical instrument includes an inserting portion 1 which may be inserted in a narrow space, such as a body cavity.

The inserting portion 1 includes a tip portion illustrated as points A1 and A2 in FIG. 1A or as a point A in FIGS. 1B and 1C. The inserting portion has an elongated cylindrical shape as illustrated. Hereafter, the side of the point A will be referred to as a tip side and the opposite side will be referred to as a base end side.

The inserting portion 1 may be used as an endoscope in which an image pickup unit, an illuminating unit and the like are mounted at the tip portion thereof or may be used as an electrophysiological catheter in which an electrode is disposed at the tip portion thereof.

If the inserting portion 1 is used as an endoscope which includes an image pickup optical system at the tip thereof, the tip includes a portion for taking light information of an object. The image pickup optical system which takes the light information may be, for example, an objective lens, optical fiber and a light transmission window for observation.

Light guided by the image pickup optical system of the endoscope is picked by an image pickup element disposed inside or outside of a medical instrument body. It is also possible to provide an image pickup element, such as a semiconductor image sensor, at the tip and perform image pickup at the tip portion.

The illuminating unit of the endoscope may use light which is emitted from a light source disposed inside or outside of the medical instrument body and is guided by, for example, optical fiber. Alternatively, the illuminating unit may include, for example, an LED at the tip thereof for illumination.

The control wires 4A and 4B are fixed at the points A1 and A2 at one ends in FIG. 1A and are connected to driving pulleys 6A and 6B at the other ends. The control wires 4A and 4B are wire materials which are bendable and by which driving force, such as tension, may be transmitted.

The control wires may be, other than wire materials which transmit tension, an electrical device of which longitudinal dimension is changed by a current.

The control wires pass through the inserting portion 1 as illustrated by the broken lines. Unillustrated guide holes are formed in the inserting portion 1 at which the portion of the control wires 4A and 4B illustrated by the broken lines so that the control wires 4A and 4B may be moved in the longitudinal direction thereof.

The positions at which the control wires 4A and 4B pass are not aligned with the center of the inserting portion 1. Being not aligned with the center of the inserting portion 1 means being disposed outside of the center of the section of the inserting portion 1. The wires may be disposed on a surface of the inserting portion.

The driving pulleys 6A and 6B are connected to clutch portions 8A and 8B. Especially the driving pulleys 6A and 6B and the clutch portions 8A and 8B constitute the drive transmitting units 2A and 2B which may transmit driving force and may stop the supply of driving force which will be described later. The clutch portions 8A and 8B are further connected to driving sources 9A and 9B. In this manner, the driving force from the driving sources 9A and 9B are transmitted to the control wires 4A and 4B via the drive transmitting units 2A and 2B.

The driving force may be, for example, tractive force to draw the wires, or a current with which the wires themselves are deformed. Hereafter, the driving force applied to the wires may be referred to as tractive force.

The inserting portion 1 includes a bending portion 3 which is a deformable portion and a non-bending portion 5. The bending portion 3 is a portion which is bent by the control wires 4A and 4B. The non-bending portion 5 is a portion which is not bent even when the control wires 4A and 4B are drawn.

As illustrated, the bending portion 3 is disposed at the tip end side and the non-bending portion 5 is disposed at the base end side. The non-bending portion may be a rigid portion which is hardly deformed or may be a bendable flexible portion (rigidity in the bending direction is greater than that of the bending portion 3).

When a signal is greater than a threshold value, the drive control unit causes the drive transmitting unit to operate to stop the supply of the driving force to the wires. The threshold value may be set in consideration of cutting strength of the control wires or a limit value of pressure application to peripheral structures.

When the supply of the driving force is stopped, the inserting portion is put into a natural state before the control wires are drawn, i.e., put into a state in which the bending portions may be easily bent. Therefore, damage to the medical instrument may be reduced.

If a plurality of control wires and a plurality of driving mechanisms which independently draw the plurality of control wires are provided, each of the driving mechanisms includes a driving force transmitting unit.

The drive control unit may send instructions to all the drive transmitting units. Therefore, the inserting portion in a state in which driving force is applied to a plurality of control wires may be put into a natural state at once in which driving force is not applied to all the control wires.

Since the operation to apply the driving force to a plurality of control wires simultaneously may control slack of the control wires inside the inserting portion and may keep the posture of the inserting portion, operability of the medical instrument is improved.

Next, a bending operation of the medical instrument according to the present embodiment will be described with reference to FIGS. 1B and 1C.

As illustrated in FIG. 1B, the driving pulley 6A draws the control wire 4A in the direction of an arrow F. The control wire 4A is fixed to the tip portion point A1 as illustrated in FIG. 1A.

The position at which the control wire 4A passes is not aligned with the center of the inserting portion 1. Therefore, tension produced when the control wire 4 is drawn becomes torque which causes the bending portion 3 to be bent in the direction of an arrow E. The bending portion 3 is bent as illustrated due to the bending torque.

The size of the bending torque may be controlled by controlling an amount of rolling up of the driving pulley 6A. In this manner, the bending operation of the bending portion 3 may be controlled. The same operation may be performed to the control wire 4B using the driving source 9B.

As illustrated in FIG. 1C, the bending portion 3 may be bent in the direction of an arrow G by drawing the control wire 4B in the direction of an arrow H. As described above, the inserting portion 1 includes two series of control wires, drive transmitting units and driving sources. By driving each of these components independently, the bending portion 3 may perform the bending operation.

A tactile sensor 7 which detects contact with, for example, peripheral structures of the tip portion is attached to the tip portion of the inserting portion 1. This is an example of a load detecting unit 22 which detects the load applied to the inserting portion 1. The load detecting unit 22 may be implemented also by other means as will be described later.

Next, a configuration of the entire medical instrument according to the present embodiment will be described with reference to FIG. 2.

The load detecting unit 22 which detects the load applied to the inserting portion 1 sends load information 101 to a controller 10 which is a control unit. The controller 10 controls the entire medical system.

During a normal operation, the controller 10 calculates a driving control signal 103 to a target position of the tip portion and instructs the same to a driving circuit 12. In accordance with the instruction, the driving circuit 12 sends driving signals 104 and 105 to the driving sources 9A and 9B, respectively.

In accordance with the instruction, the driving sources 9A and 9B operate independently. Each of the driving sources 9A and 9B transmits tractive force 108 and 109 to the drive transmitting units 2A and 2B. The drive transmitting units 2A and 2B include two states: a connected state and a disconnected state.

In the connected state which is a state of the normal operation, the control wires 4A and 4B are drawn as illustrated by tractive force 110 and 111. In this manner, as illustrated in FIGS. 1A to 1C, the control wires 4A and 4B cause the bending portion 3 to be bent.

The controller 10 monitors output of the load detecting unit 22 and determines whether the output is equal to or smaller than a threshold value 102 at which dynamic load at the tip of the inserting portion is tolerated.

If the output does not exceed the threshold value 102, transmitting portion control signals 106 and 107 which put the drive transmitting units 2A and 2B into the connected state are sent. If the output exceeds the threshold value 102, the transmitting portion control signals 106 and 107 which put the drive transmitting units 2A and 2B into the disconnected state are sent.

In accordance with the instructions, the drive transmitting units 2A and 2B are put into the disconnected state. Then transmission of the driving force to the control wires 4A and 4B is disconnected. Therefore, the control wires 4A and 4B are put into the state before being drawn. In this manner, the bending portion is put into the natural state in which it may be easily bent by external force.

The inserting portion may be removable. In that case, a portion of the inserting portion 1 enclosed with a broken line in FIG. 2 is provided separately. In that case, the separated portion may be connected with the body by connecting a wire included in the inserting portion.

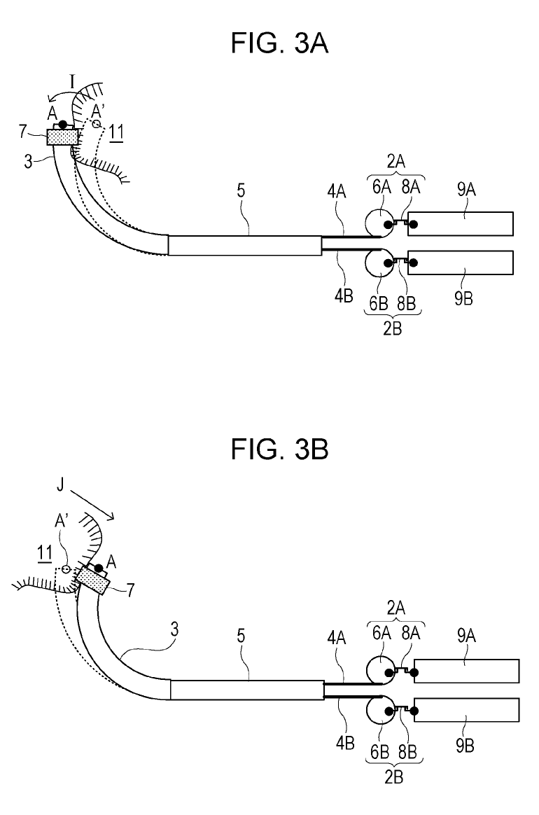

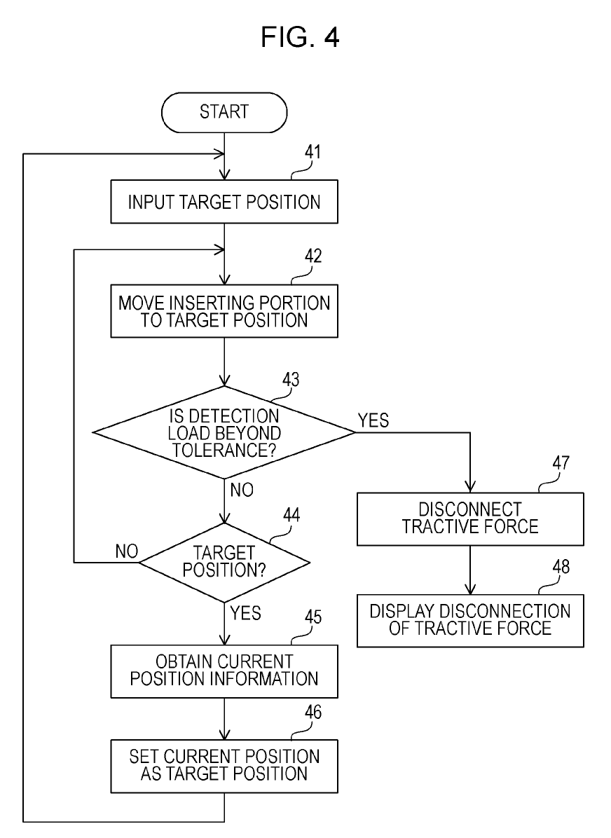

Next, an operation when the output of the load detecting unit 22 exceeds the threshold value 102 will be described in detail with reference to FIGS. 3 and 4.

FIG. 3A illustrates a state in which guidance of the inserting portion to the target position has not been performed precisely and the inserting portion 1 has been in contact with an environment 11 which is, for example, the peripheral structure. The tip portion should be the position of the point A′ but is at the position of the point A because the inserting portion is in contact with the environment 11 and is pressed in the direction of an arrow I. A drive control unit 31 controls the driving sources 9A and 9B so that the tip portion becomes the position of the point A′.

Therefore, larger load than usual is applied to the tip portion. Then large tension is applied also to the control wire 4A. Such a state may be observed by the load detecting unit 22.

In the example illustrated in FIG. 3A, the load detecting unit 22 is the tactile sensor 7 attached to the tip portion. The tactile sensor 7 detects the force received from the environment 11 and transmits the force to the controller 10.

The behavior of the medical instrument at this time will be described with reference to a flowchart of FIG. 4.

The target position is input from an input device (not illustrated) which is connected to the controller 10 (step 41). Then, the drive control unit 31 transmits, to the driving unit 21, an instruction to cause the tip portion to be moved to the target position and the driving unit 21 drives the inserting portion 1 (step 42).

When the load information 101 of the load detecting unit 22 is output to the controller 10, the load information 101 is calculated by an overload determination unit (not illustrated) inside the controller and is compared with the threshold value 102 (step 43).

If the tip portion is not in contact with the environment 11 initially, since the load information 101 of the load detecting unit 22 is equal to or smaller than the threshold value 102, the controller 10 compares the load information 101 with information of an inserting portion position detecting unit (not illustrated) and determines whether the tip portion has arrived at the target position (step 44).

Here, the inserting portion position detecting unit calculates the shape of the bending portion and the position of the tip portion on the basis of a driving amount of an encoder constituted inside the driving unit 21. The encoder may be attached to the driven pulleys 6A and 6B or the driving sources 9A and 9B.

The driving amount of the control wires 4A and 4B may be computed on the basis of the driving amount. The shape of the bending portion 3 is calculated on the basis of the driving amount of the control wires 4A and 4B.

When the tip portion arrives at the target position, a current position is obtained (step 45) and the current position is set to be the target position (step 47). In this manner, the position may be kept until the next target position input is performed by a user.

When the tip portion is brought into contact with the environment 11 and it is determined that the load information 101 exceeds the threshold value 102 in step 43, the controller 10 instructs the drive transmitting units 2A and 2B to be put into the disconnected state (step 47).

The controller 10 displays on an output device (not illustrated) that the drive transmitting units 2A and 2B are put into the disconnected state (step 48). In this manner, when the medical instrument is put into the state illustrated in FIG. 3A, the medical instrument may disconnect the tractive force to the control wires 4A and 4B and put the inserting portion into the natural state at once in which the inserting portion may be bent easily.

Therefore, cutting of the control wires 4A and 4B caused by the overload may be avoided. In the natural state, the control wires 4A and 4B are moved by very small load so that the tip portion arrives at the position of the point A.

Then, the user may extract the medical instrument as needed. Extraction may be performed in the natural state. Alternatively, the user may check that the load information 101 has become equal to or smaller than the threshold value 102 and put the drive transmitting units 2A and 2B into the connected state again, and may extract the medical instrument while operating the deformable portion.

Next, an operation in a case in which overload is applied to the tip portion of the inserting portion when the environment 11 is moved due to a certain change of state while the position is fixed will be described with reference to FIG. 3B.

The inserting portion of FIG. 3B repeats steps 41 to 47 of FIG. 4 in the state of keeping the target position. The environment 11 has moved in the direction of an arrow J. The driving sources 9A and 9B are controlled so that the tip portion is kept at the point A′. Therefore, the control wire 4B receives additional load due to the movement of the environment 11 in the direction of the arrow J. At the same time, the environment 11 is pressed by the tip portion and receives additional force.

The additional load may be detected by the tactile sensor 7 which is the load detecting unit 22 as in the case illustrated in FIG. 3A. If the load information 101 exceeds the threshold value 102, the controller 10 performs steps 43, 47 and 48 of FIG. 4 to disconnect the tractive force of the control wires 4A and 4B.

The inserting portion may be put into the natural state at once in which the insert portion may be bent easily. Therefore, cutting of the control wires 4A and 4B caused by the overload may be avoided. In the natural state, the control wires 4A and 4B are moved by very small load so that the tip portion arrives at the point A.

Therefore, if the position of the environment 11 has been changed, a retreat operation of the tip portion to a necessary direction may be performed. Then, the user may extract the medical instrument as needed. Extraction may be performed in the natural state.

Alternatively, the user may check that the load information 101 has become equal to or smaller than the threshold value 102 and put the drive transmitting units 2A and 2B into the connected state again, and may extract the medical instrument while operating the deformable portion.

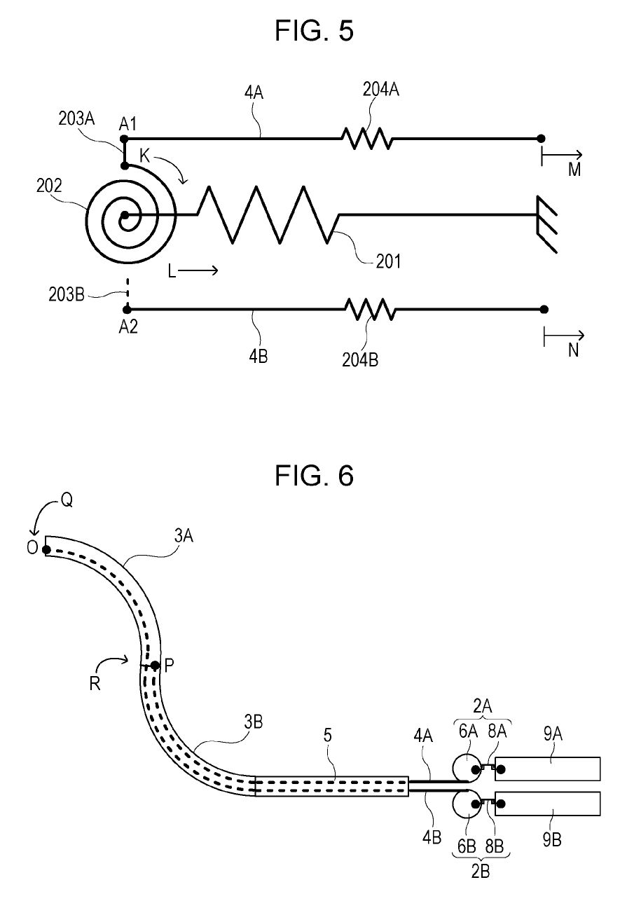

Next, an effect of the driving unit 21 which causes the a plurality of control wires 4A and 4B to operate independently will be described with reference to FIGS. 5 and 6.

FIG. 5 illustrates a simulation model in which the bending portion 3 and the control wires of FIGS. 1A to 1C are simulated by spring elements. Elasticity of the bending portion 3 is simulated by a bending spring element 202 in the bending direction K and an axial direction spring element 201 in the longitudinal direction L. These elements of the bending portion 3 are fixed at their ends. The control wires 4A and 4B are fixed independently at the points A1 and A2 at the tip portion. The distance between the point A1 and the center of the bending portion 3 is a moment arm 203A. The distance between the point A2 and the center of the bending portion 3 is a moment arm 203B. Then the control wires in the elongation direction of the wires are simulated independently by spring elements 204A and 204B.

Endpoints of the control wires 4A and 4B on the opposite side of the points A1 and A2 are drawn as illustrated by arrows M and N. Then, tractive force becomes the torque in the bending direction K (or in the reverse direction) by the moment arms 203A and 203B and the bending portion 3 may be bent.

Here, the bending curvature κ and the control wire moved amount of the bending portion 3 when the control wires are drawn will be considered. The moved amount of the control wires 4A and 4B is set to be ΔL1 and ΔL2, respectively.

Hereinafter, only the case of the control wire 4A is drawn will be described. ΔL1 is expressed by the sum of the moved amount ΔLb1 by bending displacement to the direction of an arrow K and the moved amount ΔLa1 by the axial direction displacement to the direction of an arrow L, and the moved amount ΔLt1 by expansion and contraction of the control wire itself.

ΔL1=ΔLb1+ΔLa1+ΔLt1 (Equation 1)

ΔLb1 is expressed by the following Equation 2 on the basis of the relationship of the bending curvature κ when the length of the bending portion is set to be Lb and the moment arm 203A is set to be d1.

ΔLb1=Lb·κ·d1 (Equation 2)

Here, since the moment arm 203B is opposite (=−d1) under the same bending curvature κ, the moved amount ΔLb2 of the control wire 204B of the opposite side due to bending displacement is expressed as follows.

ΔLb2=Lb·κ·(−d1) (Equation 3)

If the spring elements 201, 204A and 204B in the longitudinal direction of the bending portion 3 or the control wires 4A and 4B are ignored, the moved amount of the two control wires 4A and 4B are opposite in direction and are equal in size with respect to the bending curvature κ as described above.

In such a situation, the bending operation may be performed by, for example, winding two control wires 4A and 4B around a single pulley and causing the pulley to rotate.

Actually, the bending portion 3 and the control wires 4A and 4B include the spring elements 201, 204A and 204B. That is, the moved amount due to the bending of the control wires 4A and 4B is accompanied with offset of the moved amount like ΔLa1 and ΔLt1. Therefore, in a system in which two control wires are driven by a single pulley, precision of the position of the tip portion may be reduced or slack may occur in the control wires.

In the medical instrument according to the present embodiment, the control wires 4A and 4B are independently drawn by the driving units 21. Therefore, when the bending operation of the bending curvature κ is operated, driving in consideration of the offset of the moved amount like ΔLa1 and ΔLt1 may be performed.

The medical instrument according to the present embodiment is desirable because the control wires may be driven with high control precision at the position of the tip portion and with less production of slack.

Hereinafter, a relationship about the bending curvature κ and the control wire moved amount ΔL1 when all the three terms of the right side of Equation 1 are further considered will be described.

If distortion of the bending portion in the axial direction L is set to be Ea and distortion of the control wire 4A in the elongation direction is set to be Et, ΔLa1 and ΔLt1 are expressed as follows.

ΔLa1=Lb·εa (Equation 4)

ΔLt1=Lb·εt (Equation 5)

When the spring element with respect to the bending curvature κ of the bending spring element, the spring constant in the axial direction and the spring constant of the control wire spring element are expressed as Kb, Ka and Kt, respectively, the relationship of tension T1 applied to the control wire 4A is expressed as follows.

Kb·κ=d1·T1 (Equation 6)

Ka·εa=T1 (Equation 7)

Kt·εt=T1 (Equation 8)

Equation 1 may be expressed by tension T as follows from the above-described relationship.

The length of the control wire 4A is set to be Lt.

ΔL1=(Lb·d1̂2/Kb+Lb/Ka+Lt/Kt)·T1 (Equation 9)

Therefore, the moved amount of the control wire 4A and the bending curvature κ may be obtained from Equations 6 and 9. It is possible to control the position of the tip portion in consideration of displacement in the axial direction L by considering Equation 7. The same relationship may be obtained also for the control wire 4B.

Slack of the wires may be controlled by independently driving the control wires 4A and 4B and, therefore, positional accuracy is increased. It is also possible to perform the same operation as expressed by Equations 6 and 7 when the tension T1 is controlled.

Further, if the moved amount or tension of the control wires 4A and 4B may be measured on the basis of the relationship of Equations 6, 7 and 9, the position of the tip portion may be estimated on the basis of Lb, κ and εa.

Influences of the spring elements 201, 204A and 204B described above need to be considered especially when the outermost diameter of the inserting portion is small. The influences are significant when channels in which tools, such as a treatment tool and an endoscope, are inserted are provided inside the inserting portion.

This is for the following reason. The smaller the outermost diameter, the shorter the length of the moment arms 203A and 203B and, therefore, the smaller the outermost diameter, the smaller the bending torque with respect to the tension of the control wires. Therefore, there is a tendency that the force applied in the direction of the spring elements 201, 204A and 204B is large.

Further, if the inserting portion is thin because of the channels provided therein, the cross-sectional areas of the control wires 4A and 4B which may pass the inserting portion are small. Therefore, effects of expansion and contraction by the spring elements 204A and 204B of the control wires are significant.

The smaller the outermost diameter of the inserting portion, the more easily the inserting portion is inserted. The larger the tool channels, the greater the use of the treatment or the diagnostic tool. In the medical device according to the present embodiment, such a narrow-diameter and thin cylindrical inserting portion may be driven highly precisely. Thus, cutting of the control wires may be avoided.

Next, another effect of the driving unit 21 which causes a plurality of control wires 4A and 4B to operate independently will be described with reference to FIG. 6. In FIG. 6, two series of control wires 4A and 4B to driving sources 9A and 9B are independently provided as illustrated in FIG. 1.

The inserting portion includes two bending portions: bending portions 3A and 3B. The control wire 4A is fixed at a point P and may cause the bending portion 3B to be bent. The control wire 4B is fixed at a point O and may cause both the bending portions 3A and 3B to be bent.

Therefore, the bending portions 3A and 3B may be bent in a desired bending amount by adjusting the tension of each of the control wires 4A and 4B. As illustrated, the bending portions 3A and 3B may be bent in the opposite directions from each other.

If the bending amount of the bending portion 3B is to be changed from the illustrated state while fixing the bending amount of the bending portion 3A, it is necessary to adjust the length of the control wire 4A and, at the same time, to adjust the length of the control wire 4B. If a plurality of bending portions are disposed along the longitudinal direction of the inserting portion, coupling of the bending torque by the control wires with paths (length) of the control wires is caused between the bending portions.

Therefore, by driving a plurality of control wires 4A and 4B independently, decoupling of the torque from the paths may be performed between the bending portions. Thus, the bending amount of a plurality of bending portions 3A and 3B may be controlled independently.

If a plurality of bending portions are provided, the inserting portion may be guided with lower invasiveness to complicated and narrow body cavities. A procedure therefor performed by a doctor may become easier.

### Second Embodiment

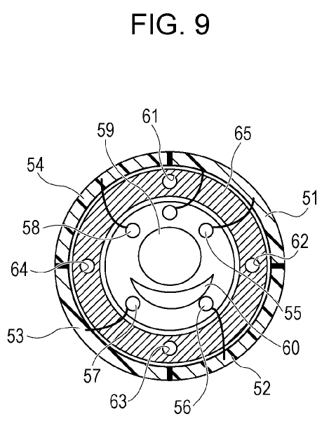

FIG. 9 illustrates a tactile sensor 7 which is an example of the load detecting unit 22 in the block diagram of FIG. 2. FIG. 9 is a vertical cross-sectional view of the tip portion of the inserting portion 1 along the longitudinal direction of FIG. 1. The inserting portion 1 includes a sheath 65 which is a cylindrical structure as a body.

The tactile sensor 7 is made of a conductive resin material which has four areas 51, 52, 53 and 54 on a surface of the sheath 65 along the circumferential direction of the sheath 65. Resistance values of the four areas are changed in accordance with load applied thereto. Detected values, i.e., the amount of change of resistance, of each area are measured.

The direction and the value of the applied load may be computed by calculating in an internal computing unit (not illustrated) of the controller 10. Output of the tactile sensor 7 is transmitted to the controller 10 by conductive members 55, 56, 57 and 58 which pass through the inserting portion 1.

Here, the reference sign 59 denotes an optical fiber bundle for image observing and 60 denotes optical fiber for illumination. 61, 62, 63 and 64 denote guide holes in which the control wires are inserted. These guide holes are disposed outside the center of the section of the inserting portion.

By providing the tactile sensor 7 at the tip portion, the force at the location at which the overload has occurred in accordance with the relationship with the environment 11 as described above may be observed directly. The detected load information 101 is highly precise and is less easily affected by the disturbance.

Therefore, if the inserting portion 1 is put into an overloaded state, it is possible to change the state of the drive transmitting units 2A and 2B to the disconnected state.

By providing the tactile sensor 7 at the tip portion of the inserting portion 1, the load applied to the portion with the highest possibility of being brought into contact with the environment 11 may be observed.

### Third Embodiment

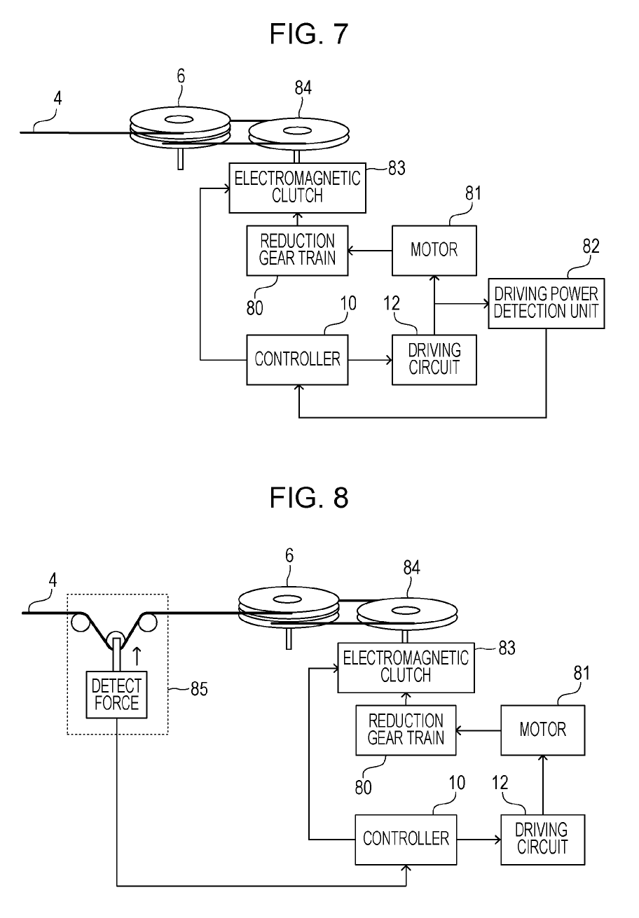

A third embodiment of the present invention will be described with reference to FIG. 7. Components having the same functions as those of the second embodiment will be denoted by the same reference signs and description thereof will be omitted. FIG. 7 is a schematic block diagram illustrating a configuration of each of series of the control wires 4A and 4B to the driving sources 9A and 9B of the medical instrument of FIG. 1.

The present embodiment differs from the second embodiment in that the load detecting unit 22 is not the tactile sensor 7 but a driving current detection unit 82.

Hereinafter, the control wires 4A and 4B will be collectively referred to as a control wire 4 and the driven pulleys 6A and 6B will be collectively referred to as a driving pulley 6. Each of the two driving systems has the same configuration which will be described below.

Each component is controlled by a controller which is a control unit.

In the present embodiment, a motor 81 and a reduction gear train 80 are provided as driving sources 9A and 9B. The reduction gear train 80 transmits power to an electromagnetic clutch 83. The electromagnetic clutch 83 is connected to a round connecting unit 84.

The electromagnetic clutch 83 and the round connecting unit 84 correspond to the clutch portions 8A and 8B of FIGS. 1A to 1C. The round connecting unit 84 may transmit power to the driving pulley 6.

In this manner, the motor 81 may draw the control wire 4 in accordance with the driving signal from the driving circuit 12. The electromagnetic clutch 83 may connect and disconnect power in response to the instructions from the controller 10.

The driving current detection unit 82 may detect a driving current of the motor 81.

When the load applied to the control wire 4 is increased, the driving current becomes large. Therefore, the load applied to the control wire 4 may be detected by detecting the driving current in the driving current detection unit 82.

The detection signal of the driving current is transmitted to the controller 10. The size of the applied load may be computed in an internal computing unit (not illustrated). However, it is not necessary to calculate the load. As described above, the load detecting unit 22 may be implemented by also using the driving current detection unit 82 provided in the driving unit 21.

By using the driving current detection unit 82, load applied to the inserting portion 1 may be detected without providing any special configuration in the inserting portion 1. Therefore, the size of the inserting portion 1 may be reduced and thus channels for large-sized treatment tools may be provided inside the inserting portion 1.

Since the size of a current detecting sensor may be reduced, the size of the driving source may be reduced. This is important especially when a plurality of series of driving sources are required. Further, influences on the operation of the medical device caused by the detection of the load may be minimized.

### Fourth Embodiment

A fourth embodiment will be described with reference to FIG. 8. Components having the same functions as those of the second and the third embodiments will be denoted by the same reference signs and description thereof will be omitted. The present embodiment is the same with the third embodiment except that a tension meter 85 is used as the load detecting unit 22 instead of the driving current detection unit 82.

The tension meter 85 is disposed between the driving pulley 6 and the control wire 4 as illustrated. Since the control wire 4 is wound around three rollers, the tension of the control wire 4 may be detected as force in the direction of an arrow in a broken line of the tension meter 85.

The detected tension (load) is sent to the controller 10 and the controller 10 may compare the load with a threshold value.

By using the tension meter 85, load applied to the inserting portion 1 may be detected without providing any special configuration in the inserting portion 1. Therefore, the size of the inserting portion 1 may be reduced and thus channels for large-sized treatment tools may be provided inside the inserting portion 1.

Since the tension of the control wire 4 is measured directly, it is possible to detect the load on the inserting portion 1 without being affected by errors of other components from the motor 81 to the control wire 4 (i.e., the reduction gear train 80, the clutch 83, the round connecting unit 84 and the driving pulley 6). If the influence of expansion and contraction by the spring elements 201, 204A and 204B of FIG. 5 is significant, the load of the inserting portion 1 may be estimated without being affected by the influence.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

### REFERENCE SIGNS LIST

1 inserting portion
2, 2A and 2B drive transmitting units
3, 3A and 3B bending portions
4, 4A and 4B control wires
7 tactile sensor
10 controller
11 environment

### ADVANTAGEOUS EFFECTS OF INVENTION

According to the present invention, a medical instrument in which damage, such as cutting of a wire, caused to the medical instrument may be reduced by breaking, by a driving unit, connection between the wire and driving force even when excessively large load is applied to a deformable portion may be provided.

1. A medical instrument comprising:
a deformable portion;
a plurality of wires configured to deform the deformable portion;
a driving unit configured to generate driving force to independently drive the plurality of wires, and
a controller configured to output driving signals to control the driving unit, the driving signals being signals for which expansion or contraction of the wires due to driving of the wires is compensated.

2. The medical instrument according to claim 1, further comprising:
a driving force transmitter configured to transmit the driving force from the driving unit to the wire,
wherein the medical instrument includes a load detecting unit configured to detect load externally applied to the deformable portion; and
in a case where the load detected by the load detecting unit exceeds a threshold value, the driving force transmitter breaks connection between the wire and the driving unit, thereby putting the deformable portion to a natural state at which the deformable portion is deformable in accordance with an external force,
wherein in a case where the load detected by the load detecting unit is below the threshold value while the deformable portion is in the natural state, the driving force transmitter resumes connection between the wires.

3. The medical instrument according to claim 1, wherein the wires inserted at a position outside the center of a section of the deformable portion.

4. The medical instrument according to claim 3, wherein the plurality of wires are disposed to surround the center of the section of the deformable portion.

5. The medical instrument according to claim 1, further comprising a calculating unit configured to calculate the load using a current value of a driving current which flows through the driving unit.

6. The medical instrument according to claim 2, wherein the driving unit breaks connection between the wire and the driving force in a manner in which the wires are not destroyed.

7. The medical instrument according to claim 1, further comprising an image pickup unit and an illuminating unit at a tip of the deformable portion.

8. The medical instrument according to claim 1, further comprising a light source configured to supply illumination light to the illuminating unit and a light guide unit configured to guide the illumination light from the light source.

9. The medical instrument according to claim 1, wherein the illuminating unit is a light-emitting device array.

10. The medical instrument according to claim 2, wherein the load detecting unit is a measuring unit configured to measure pressure and the load detecting unit is disposed at the tip of the deformable portion.

11. The medical instrument according to claim 2, wherein the load detecting unit is a measuring unit configured to measure a current for driving the driving unit.

12. The medical instrument according to claim 2, wherein the load detecting unit is a measuring unit configured to measure tension applied to the wires.

13. The medical instrument according to claim 12, wherein the load detection unit measures tension of the wires between the deformable portion and the driving unit.

14. The medical instrument according to claim 2, wherein the driving unit decreases the tension applied to the wire to zero.

15. The medical instrument according to claim 4, wherein the driving unit simultaneously decreases the tension applied to the plurality of wires.

16. The medical instrument according to claim 2, wherein the controller causes a presenting device to present to an operator of the instrument, information that the connection between the wire and the driving unit is broken.

17. The medical instrument according to claim 1, further comprising:
a plurality of the motors, which drive mutually different one of the plurality of wires.

18. The medical instrument according to claim 17, comprising a plurality of the deformable portions each of which associated with mutually different ones of the plurality of the wires.
